(19) United States
(12) Patent Application Publication
(10) Pub. No.: US 2023/0218268 A1
(43) Pub. Date: Jul. 13, 2023

(54) METHOD AND SYSTEM FOR DETECTING LOCATION OF A SEGMENT OF A FEEDING TUBE INSIDE A PATIENT

(71) Applicants: Giant Power Technology Biomedical Corp., New Taipei City (TW); National Taipei University of Technology, Taipei City (TW)

(72) Inventors: Ming-Kun Huang, New Taipei City (TW); Chien-Jen Wang, New Taipei City (TW); Po-En Liu, Taipei City (TW); Shu-Hung Chao, New Taipei City (TW); Ing-Lan Liou, New Taipei City (TW); Chun- Young Chang, Taipei City (TW); Chin-Kun Tseng, New Taipei City (TW); Zi-Yi Zhuang, New Taipei City (TW); Ya-Wen Chao, New Taipei City (TW); Hsuan-Yu Liu, New Taipei City (TW); Gu-Neng Wu, New Taipei City (TW); Chun-Ling Lin, New Taipei City (TW); Yuh-Shyan Hwang, Taipei City (TW); San-Fu Wang, New Taipei City (TW); I-Chyn Wey, New Taipei City (TW); Jason King, New Taipei City (TW)

(21) Appl. No.: 17/831,723

(22) Filed: Jun. 3, 2022

(30) Foreign Application Priority Data
Jan. 11, 2022 (TW) 111101143

(51) Int. Cl. A61B 8/00; A61J 15/00; A61B 8/12

(52) U.S. Cl. CPC A61B 8/4254; A61J 15/0003; A61J 15/0088; A61B 8/12

(57) ABSTRACT

A method for detecting a location of a segment of a feeding tube is provided. The feeding tube has a proximal end, a hollow tube body and a distal end, and is placed inside the body of a patient. An audio collecting component is placed on a predetermined part of the patient. The method includes steps of pumping air into the proximal end of the feeding tube, collecting sound to obtain audio data by the audio collecting component, performing audio analysis on the audio data, and determining whether a segment of the hollow tube body is at a part inside the body of the patient that corresponds with the location of the audio collecting component based on result of the audio analysis.

### CROSS-REFERENCE TO RELATED APPLICATION

This application claims priority of Taiwanese Patent Application No. 111101143, filed on Jan. 11, 2022.

### FIELD

The disclosure relates to a method and a system for detecting a location of a segment of a feeding tube inside a patient.

### BACKGROUND

Typically, a feeding tube may be employed to help provide liquid and/or nutrition to patients having difficulty swallowing. Placement of the feeding tube is typically done by inserting one end of the feeding tube (known as a distal end) through the nasal cavity of the patient, and through the esophagus into the stomach or in a section of the small intestine. After the placement is complete, nutrition may be provided from another end (known as a proximal end) into the stomach or the section of the small intestine of the patient. However, since the person who performs the placement of the feeding tube (e.g., medical personnel) is unable to clearly see the inside of the patient during the process (also known as a blind insertion), misplacement may occur where the distal end of the feeding tube ends up in, for example, a part of the respiratory tract of the patient.

### SUMMARY

Therefore, an object of the disclosure is to provide a method that can detect a location of a segment of a feeding tube.

According to one embodiment of the disclosure, the method, the feeding tube has a proximal end, a hollow tube body and a distal end and being disposed inside a body of a patient. The method is implemented using a system that includes a processing device, an air pumping device and an audio collecting module. The audio collecting module includes a first audio collecting component that is placed on a first predetermined part of the patient. The method includes:

by the air pumping device, pumping air into the proximal end of the feeding tube;

by the first audio collecting component, collecting sound related to the first predetermined part of the patient to obtain first audio data;

by the processing device, performing audio analysis on the first audio data obtained by the first audio collecting component; and

by the processing device, determining whether a segment of the hollow tube body is at a part inside the body of the patient that corresponds with the location of the first audio collecting component based on a result of the audio analysis performed on the first audio data.

Another object of the disclosure is to provide a system that is configured to implement the above-mentioned method.

According to one embodiment of the disclosure, the feeding tube has a proximal end, a hollow tube body and a distal end. The distal end of the feeding tube is inserted, via the nasal cavity, into the body of a patient.

The system includes a processing device, an air pumping device, and an audio collecting module that includes a first audio collecting component placed on a first predetermined part of the patient.

The air pumping device is operable to pump air into the proximal end of the feeding tube.

The first audio collecting component is configured to collect sound related to the first predetermined part of the patient to obtain first audio data; and

The processing device is configured to perform audio analysis on the first audio data obtained by said first audio collecting component, and to determine whether a segment of the hollow tube body is at a part inside the body of the patient that corresponds with the location of said first audio collecting component based on a result of the audio analysis performed on the first audio data.

### BRIEF DESCRIPTION OF THE DRAWINGS

Other features and advantages of the disclosure will become apparent in the following detailed description of the embodiments with reference to the accompanying drawings, of which:

FIG. 1 is a block diagram illustrating a system for detecting a location of a segment of a feeding tube according to one embodiment of the disclosure;

FIG. 2 is a flow chart illustrating steps of a method for detecting a location of a segment of a feeding tube according to one embodiment of the disclosure; and

FIG. 3 illustrates the use of the system on the patient.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

Throughout the disclosure, the term “connected to” may refer to a direct connection among a plurality of electrical apparatus/devices/equipments via an electrically conductive material (e.g., an electrical wire), or an indirect connection between two electrical apparatus/devices/equipments via another one or more apparatus/device/equipment or wireless communication.

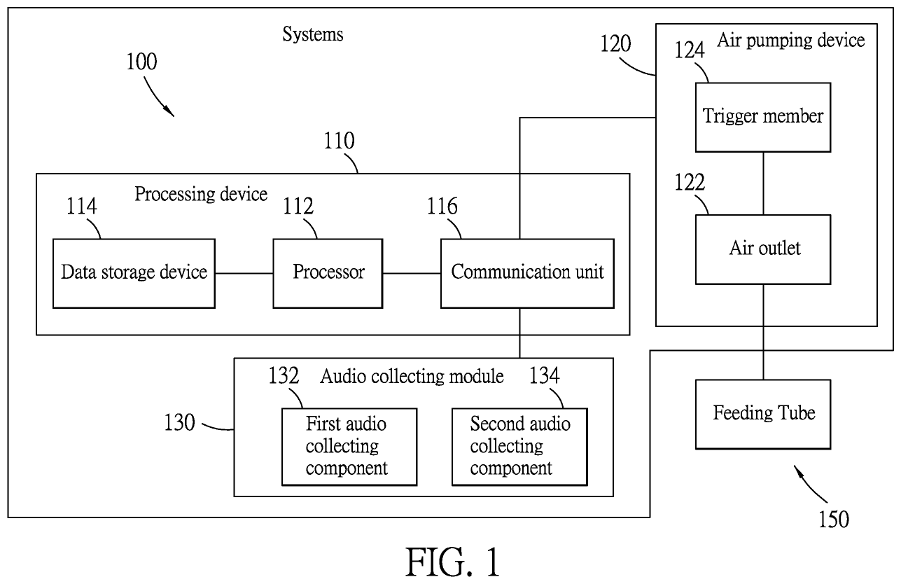

FIG. 1 is a block diagram illustrating a system 100 for detecting a location of a segment of a feeding tube 150 according to one embodiment of the disclosure.

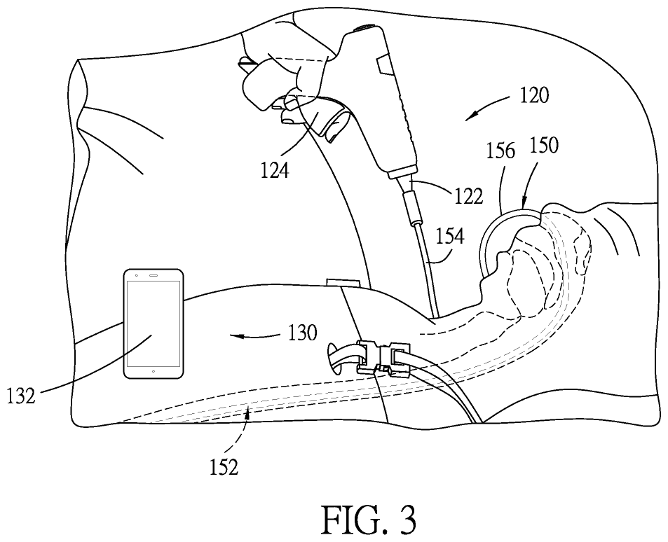

In this embodiment, the feeding tube 150 is to be placed in a patient for the purpose of providing nutrition to the patient. Referring to FIG. 3, in use, the placement of the feeding tube 150 is typically done by inserting one end of the feeding tube (known as a distal end 152) through the nasal cavity of the patient, and through the esophagus into the stomach or in a section of the small intestine. After the placement is complete, nutrition may be provided from another end (known as a proximal end 154) into the stomach or the section of the small intestine of the patient. The feeding tube 150 includes a hollow tube body 156 having openings at the distal end 152 and the proximal end 154, respectively.

The above placement is typically done without visual of the inside of the patient (also known as a blind insertion). In such cases, part of the hollow tube body 156 may end up in unintended parts of the patient such as, parts of the respiratory tract of the patient. Such an incident is referred to as misplacement of the feeding tube 150.

As a result, in this embodiment, the system 100 is employed to detect a location of a segment of the feeding tube 150 during the placement of the feeding tube 150 or after the placement is done. Specifically, the system 100 includes a processing device 110, an air pumping device 120 and an audio collecting module 130.

The processing device 110 may be embodied using a personal computer, a laptop, a tablet, a smartphone, etc., and includes a processor 112, a data storage device 114 and a communication unit 116.

The processor 112 may include, but not limited to, a single core processor, a multi-core processor, a dual-core mobile processor, a microprocessor, a microcontroller, a digital signal processor (DSP), a field-programmable gate array (FPGA), an application specific integrated circuit (ASIC), and/or a radio-frequency integrated circuit (RFIC), etc.

The data storage device 114 may be embodied using one or more of a hard disk, a solid-state drive (SSD) and other non-transitory storage medium, and stores a software application therein.

The communication unit 116 may include one or more of a radio-frequency integrated circuit (RFIC), a short-range wireless communication module supporting a short-range wireless communication network using a wireless technology of Bluetooth® and/or Wi-Fi, etc., and a mobile communication module supporting telecommunication using Long-Term Evolution (LTE), the third generation (3G) and/or fifth generation (5G) of wireless mobile telecommunications technology, and/or the like.

The air pumping device 120 includes an air outlet 122 and a trigger member 124. FIG. 3 illustrates one exemplary form of the air pumping device 120, which is roughly in the shape of a nail gun.

In some embodiments, the air pumping device 120 may be embodied using a syringe, with a bevel of the syringe serving as the air outlet 122, and a plunger of the syringe serving as the trigger member 124.

In some embodiments, the air pumping device 120 may be embodied using an electrical air pump that is capable of supplying the air to be pumped. In embodiments, pumping of the air may be controlled using the trigger member 124 (such as a switch, a lever mechanism, etc.) that is movable between an engaged position, where the air pumping device 120 is controlled to pump air out from the air outlet 122, and an unengaged position, where the air pumping device 120 is controlled to stop pumping air.

The trigger member 124 may be operated manually or by the processor 112 executing the software application. In the latter case, the air pumping device 120 may be connected to the processing device 110 via the communication unit 116.

The audio collecting module 130 is connected to the processing device 110, and includes a first audio collecting component 132. In embodiments, the first audio collecting component 132 may be embodied using an audio pickup device, such as a microphone, that is capable of collecting sound to obtain audio data. The audio data may then be transmitted to the processing device 110, which is configured to perform audio analysis on the audio data. FIG. 3 illustrates one exemplary form of the audio collecting module 130, which may be a smartphone that includes a microphone for collecting sound. In some embodiments, the processing device 110 and the audio collecting module 130 may be integrated using the smartphone or other electronic devices with the similar functionalities.

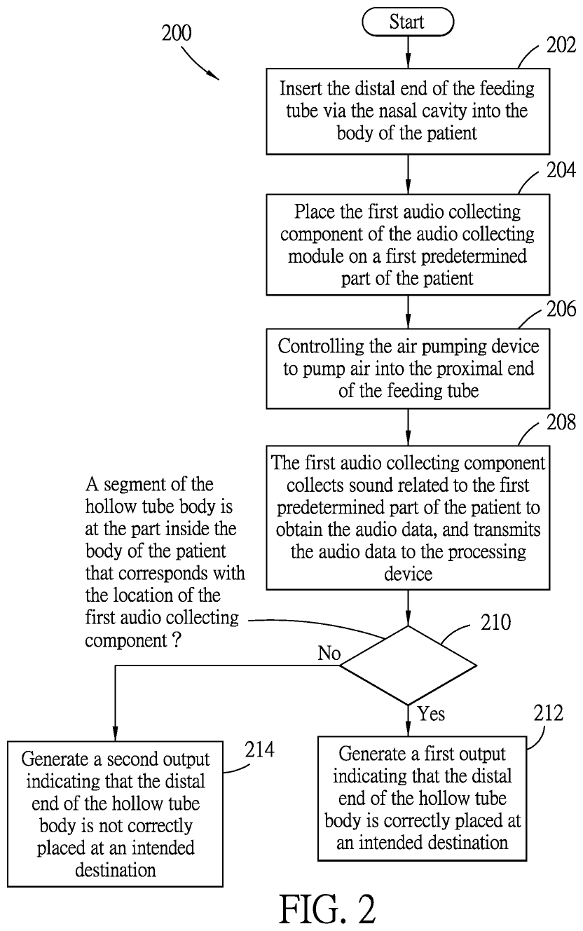

FIG. 2 is a flow chart illustrating steps of a method 200 for detecting a location of a segment of a feeding tube 150 according to one embodiment of the disclosure. In this embodiment, the method is implemented using the system 100 as shown in FIG. 1. FIG. 3 illustrates the use of the system 100 on a patient.

In step 202, the distal end 152 of the feeding tube 150 is inserted via the nasal cavity into the body of the patient. In this embodiment, the insertion may be done manually without visual of the inside of the patient.

In step 204, the first audio collecting component 132 of the audio collecting module 130 is placed on a first predetermined part of the patient. In this embodiment, the first predetermined part of the patient is a body part corresponding in position with the stomach of the patient. That is to say, the first audio collecting component 132 may be placed on the belly of the patient.

It is noted that the operations of steps 202 and 204 may be performed in arbitrary order. That is to say, in some embodiments, the operation of step 204 may be performed prior to that of step 202. In some embodiments, the operations of steps 202 and 204 may be performed simultaneously.

In step 206, the air pumping device 120 is controlled to pump air into the proximal end 154 of the feeding tube 150. Specifically, the air outlet 122 of the air pumping device 120 may be physically connected to the proximal end 154 (e.g., inserted into the proximal end 154), and the trigger member 124 may be operated to pump air into the feeding tube 150.

In the embodiments where the air pumping device 120 is embodied using the electrical air pump, the air pumping device 120 is configured to support a number of different operation modes of pumping air. For example, the air pumping device 120 may be configured to operate in one of a first mode where one shot of air is pumped for a predetermined time period when the trigger member 124 is moved to the engaged position (also known as a semi-automatic mode), a second mode where bursts of air are pumped in succession for a predetermined number of times when the trigger member 124 is moved to the engaged position (also known as a burst mode), and a third mode, where air is pumped continuously or in successive bursts when the trigger member 124 is moved to the engaged position (also known as a fully-automatic mode). In any of the operation modes, the air may be pumped at a preset frequency.

It is noted that when air is pumped into the hollow tube body 156 of the feeding tube 150, a specific form of acoustic sound will be generated owing to vibration of the hollow tube body 156. Using this characteristics, the system 100 may be configured to obtain the audio data and determine whether the specific form of acoustic sound is collected, so as to determine whether a segment of the hollow tube body 156 (e.g., a segment that is adjacent to the distal end 152) is at a part (e.g., the stomach) inside the body of the patient that corresponds with the location of the first audio collecting component 132.

In step 208, the first audio collecting component 132 collects sound related to the first predetermined part of the patient to obtain the audio data, and transmits the audio data to the processing device 110.

In step 210, in response to receipt of the audio data, the processor 112 of the processing device 110 executes the software application to perform the audio analysis on the audio data and determine whether a segment of the hollow tube body 156 is at the part inside the body of the patient that corresponds with the location of the first audio collecting component 132 based on a result of the audio analysis.

Specifically, the audio analysis may include determining a waveform of the audio data, a frequency of the audio data, a sound pressure level of the audio data, etc. It is noted that the acoustic sound of the air flowing into the hollow tube body 156 may be collected in advance, analyzed and stored in the data storage 114 for comparison with the audio data during the sound analysis process. Moreover, the processor 112 may be able to differentiate the acoustic sound of the air flowing into the hollow tube body 156 and other kinds of sound, such as the sound of peristalsis of the stomach, the sound of the small intestine, etc. In some embodiments, the acoustic sound of the air flowing out from the distal end 152 of the feeding tube 150 may also be collected in advance, analyzed and stored in the data storage 114 for comparison with the audio data during the audio analysis.

In use, the audio analysis may be performed on the audio data to determine whether air is being pumped into the hollow tube body 156 nearby the first audio collecting component 132 (and therefore, the distal end 152 of the feeding tube 150 is correctly placed in the stomach of the patient). It is noted that in this embodiment, a minimum sound pressure level (expressed using the unit of decibel) is used to determine that a segment of the hollow tube body 156 is nearby. That is to say, when it is determined by performing the audio analysis that, based on the waveform of the audio data and/or the frequency of the audio data, the acoustic sound of the air flowing into the hollow tube body 156 is detected, but the sound pressure level of the audio data is smaller than the minimum sound pressure level, the result of the audio analysis may indicate that no segment of the hollow tube body 156 is nearby the audio collecting component 132.

When it is determined that a segment of the hollow tube body 156 is at the part inside the body of the patient that corresponds with the location of the first audio collecting component 132, in step 212, the processing device 110 may generate a first output indicating that the distal end 152 of the hollow tube body 156 is correctly placed at an intended destination (e.g., the stomach in this embodiment, or small intestine in some embodiments) inside the patient. The first output may be in the form of a visual cue (e.g., a green light, a text, etc.) or an audio output (e.g., a buzz sound, a spoken phrase, etc.). In this way, relevant personnel may be notified to start providing nutrition through the feeding tube 150 into the stomach or the small intestine of the patient.

Otherwise, when it is determined that no segment of the hollow tube body 156 is at the part inside the body of the patient that corresponds with the location of the first audio collecting component 132 (for example, no audio data that corresponds with the acoustic sound of the air flowing into the hollow tube body 156 is detected, or the sound pressure level of the audio data is smaller than the minimum sound pressure level), in step 214, the processing device 110 may generate a second output indicating that the distal end 152 of the hollow tube body 156 is not correctly placed at the intended destination inside the patient. The second output may be in the form of a visual cue (e.g., a red light, a text, etc.) or an audio output (e.g., a buzz sound, a spoken phrase, etc.).

In the case that the second output is received, relevant personnel may proceed to move the first audio collecting component 132 to another part of the body of the patient (e.g., the another part may correspond to a potential misplacement location, such as the respiratory tract, so the first audio collecting component 132 is moved to be placed on the chest of the patient), and the air pumping device 120 may be operated to perform the operation of step 206. Then, the operations of steps 208 and 210 are also performed to determine whether a segment of the hollow tube body 156 is at a part inside the body of the patient that corresponds with the new location of the first audio collecting component 132, such as a lung of the patient.

It is noted that while in the previous description, only one audio collecting component is present, in some embodiments, multiple audio collecting components may be present. In one example, in step 204, a second audio collecting component 134 similar in structure to the first audio collecting component 132 is placed on a second predetermined part of the patient (e.g., the chest), and in step 208, the second audio collecting component 134 collects sound related to the second predetermined part to obtain audio data.

Subsequently, after step 208, the audio data obtained by each of the first audio collecting component 132 and the second audio collecting component 134 is analyzed, so as to determine whether a segment of the hollow tube body 156 is at the part inside the body of the patient that corresponds with the location of the first audio collecting component 132 or the part inside the body of the patient that corresponds with the location of the second audio collecting component 134.

In this case, since both the first audio collecting component 132 and the second audio collecting component 134 may capture audio data that includes the acoustic sound of the air flowing into the hollow tube body 156, the sound pressure levels of the audio data from the first audio collecting component 132 and the second audio collecting component 134 may be compared to determine whether the hollow tube body 156 is closer to the first audio collecting component 132 or the second audio collecting component 134 (i.e., the hollow tube body 156 is closer to the audio collecting component that captures a louder acoustic sound (higher sound pressure level)). Then, the processor 112 may be able to determine whether a segment of the hollow tube body 156 is correctly placed in the stomach of the patient (or the small intestine of the patient).

To sum up, the embodiments of the disclosure provide a method and a system for detecting a location of a segment of a feeding tube inside a patient. By pumping air into the hollow tube body of the feeding tube and using the audio collecting component(s) to obtain the audio data, the processor may determine whether a segment of the hollow tube body is near where each audio collecting component is located, and to deduce whether the distal end of the feeding tube is correctly placed at the intended destination. It is noted that such a configuration may be implemented to achieve the intended effect without requiring visual of the inside of the patient, and therefore may be implemented at a relatively low cost while reducing the risk of misplacement of the feeding tube.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for detecting a location of a segment of a feeding tube, the feeding tube having a proximal end, a hollow tube body and a distal end and being disposed inside a body of a patient, the method to be implemented using a system that includes a processing device, an air pumping device and an audio collecting module, the audio collecting module including a first audio collecting component that is placed on a first predetermined part of the patient, the method comprising steps of:
by the air pumping device, pumping air into the proximal end of the feeding tube;
by the first audio collecting component, collecting sound related to the first predetermined part of the patient to obtain first audio data;
by the processing device, performing audio analysis on the first audio data obtained by the first audio collecting component; and
by the processing device, determining whether a segment of the hollow tube body is at a part inside the body of the patient that corresponds with the location of the first audio collecting component based on a result of the audio analysis performed on the first audio data.

2. The method of claim 1, wherein the step of pumping air includes one of:
pumping one shot of air for a predetermined time period into the proximal end of the feeding tube;
pumping bursts of air in succession for a predetermined number of times into the proximal end of the feeding tube; and
pumping air continuously into the proximal end of the feeding tube.

3. The method of claim 1, wherein the first predetermined part of the patient corresponds in position with the stomach of the patient.

4. The method of claim 3, the audio collecting module further including a second audio collecting component that is placed on a second predetermined part of the patient, the method further comprising, subsequent to the step of pumping air, steps of:
by the second audio collecting component, collecting sound related to the second predetermined part of the patient to obtain second audio data;
by the processing device, performing the audio analysis on the second audio data; and
by the processing device, determining whether a segment of the hollow tube body is at a part inside the body of the patient that corresponds with the location of the second audio collecting component based on result of the audio analysis performed on the second audio data.

5. The method of claim 4, further comprising:
by the processing device, determining whether the hollow tube body is closer to the first audio collecting component or the second audio collecting component by comparing a sound pressure level included in the first audio data and the second audio data;
wherein the step of determining whether a segment of the hollow tube body is at a part inside the body of the patient that corresponds with the location of the first audio collecting component and the step of determining whether a segment of the hollow tube body is at the part inside the body of the patient that corresponds with the location of the second audio collecting component are done based on the determination of whether the hollow tube body is closer to the first audio collecting component or the second audio collecting component.

6. The method of claim 4, wherein the second predetermined part of the patient corresponds in position with a lung of the patient.

7. The method of claim 1, further comprising, after it is determined that a segment of the hollow tube body is at the part inside the body of the patient that corresponds with the location of the first audio collecting component:
generating an output indicating that the distal end of the hollow tube body is correctly placed at an intended destination inside the patient.

8. The method of claim 7, further comprising, after it is determined that no segment of the hollow tube body is at the part inside the body of the patient that corresponds with the location of the first audio collecting component:
generating an output indicating that the distal end of the hollow tube body is incorrectly placed.

9. The method of claim 7, wherein the step of determining whether a segment of the hollow tube body is at a part inside the body of the patient that corresponds with the location of the second audio collecting component is done based on at least a sound pressure level of the first audio data.

10. A system for detecting a location of a segment of a feeding tube, the feeding tube having a proximal end, a hollow tube body and a distal end, the distal end of the feeding tube being inserted, via the nasal cavity, into the body of a patient, the system comprising a processing device, an air pumping device, and an audio collecting module that includes a first audio collecting component placed on a first predetermined part of the patient, wherein:
said air pumping device is operable to pump air into the proximal end of the feeding tube;
said first audio collecting component is configured to collect sound related to the first predetermined part of the patient to obtain first audio data; and
said processing device is configured to perform audio analysis on the first audio data obtained by said first audio collecting component, and to determine whether a segment of the hollow tube body is at a part inside the body of the patient that corresponds with the location of said first audio collecting component based on a result of the audio analysis performed on the first audio data.

11. The system of claim 10, wherein said air pumping device includes a trigger member that is movable between an engaged position and an unengaged position, and said air pumping device is controlled to pump air in one of:
a first mode, where one shot of air is pumped for a predetermined time period when said trigger member is moved to the engaged position;
a second mode, where bursts of air are pumped in succession for a predetermined number of times when said trigger member is moved to the engaged position; and
a third mode, where air is pumped continuously when said trigger member is moved to the engaged position.

12. The system of claim 10, wherein the first predetermined part of the patient corresponds in position with the stomach of the patient.

13. The system of claim 12, wherein:
said audio collecting module further includes a second audio collecting component that is placed on a second predetermined part of the patient and that is configured to collect sound related to the second predetermined part to obtain second audio data; and
said processing device is configured to perform the audio analysis on the second audio data obtained by said second audio collecting component, and determining whether a segment of the hollow tube body is at a part inside the body of the patient that corresponds with the location of said second audio collecting component based on a result of the audio analysis performed on the second audio data.

14. The system of claim 13, wherein:
said processing device is further configured to determine whether the hollow tube body is closer to said first audio collecting component or said second audio collecting component by comparing a sound pressure level included in the first audio data and the second audio data;
said processing device is configured to determine whether a segment of the hollow tube body is at the part inside the body of the patient that corresponds with the location of said first audio collecting component and whether a segment of the hollow tube body is at the part inside the body of the patient that corresponds with the location of said second audio collecting component based on the determination of whether the hollow tube body is closer to said first audio collecting component or said second audio collecting component.

15. The system of claim 13, wherein the second predetermined part of the patient corresponds in position with a lung of the patient.

16. The system of claim 10, wherein said processing device is further configured to, after it is determined that a segment of the hollow tube body is at the part inside the body of the patient that corresponds with the location of said first audio collecting component, generate an output indicating that the distal end of the hollow tube body is correctly placed into an intended destination inside the patient.

17. The system of claim 16, wherein said processing device is further configured to, after it is determined that no segment of the hollow tube body is at the part inside the body of the patient that corresponds with the location of said first audio collecting component:
generate an output indicating that the distal end of the hollow tube body is incorrectly placed.

18. The system of claim 17, wherein said processing device is configured to determine whether a segment of the hollow tube body is at a part inside the body of the patient that corresponds with the location of said first audio collecting component based on at least a sound pressure level of the first audio data.
